(19) United States
(12) Patent Application Publication
(10) Pub. No.: US 2012/0061487 A1
(43) Pub. Date: Mar. 15, 2012

(54) Pheromone Dispersing Clothing Apparatus

(76) Inventors: Thomas J. Fortin, Wauwatosa, WI (US); Crystal M. Hess, New Berlin, WI (US)

(21) Appl. No.: 13/231,744

(22) Filed: Sep. 13, 2011

Related U.S. Application Data
(60) Provisional application No. 61/382,977, filed on Sep. 15, 2010.

(51) Int. Cl. A61L 9/04

(52) U.S. Cl. 239/36

(57) ABSTRACT

The present invention is a pheromone dispersing clothing apparatus with a body pheromone activating panel adapted to receive pheromone from a pheromone supply reservoir and a concealed compartment adapted to receive a detachable pheromone supply reservoir.

### CROSS-REFERENCE TO RELATED APPLICATION

This application claims priority to U.S. Provisional Application No. 61/382,977 filed Sep. 15, 2010.

### FIELD OF INVENTION

The present invention relates to the field of pheromone containers, and more specifically to a fabric pheromone dispersing apparatus.

### BRIEF DESCRIPTION OF THE DRAWINGS

FIG. 1 illustrates an exemplary embodiment of a pheromone dispersing apparatus at the point-of-purchase.

FIG. 2 illustrates an exemplary embodiment of a fabric pheromone dispersing apparatus for use in a garment at the point-of-purchase.

FIG. 3 illustrates an exemplary embodiment of a pheromone dispersing apparatus ready for use.

FIG. 4 illustrates an exemplary embodiment of a pheromone dispersing apparatus activated by heat generated from a personal electronic device.

FIG. 5 illustrates an exemplary embodiment of a pheromone dispersing apparatus activated by iron oxide.

FIG. 6 illustrates an exemplary embodiment of a pheromone dispersing apparatus which indicates the presence of pheromones with a thermochromatic textile area.

### GLOSSARY

As used herein, the term “garment” refers to any item of manufacture adapted to be worn by a person, including clothing, footwear and accessories.

As used herein, the term “non-irritating material” refers to any material known in the art to not cause skin irritation or to create a noticeable tactile effect.

As used herein, the term “pheromone” refers to any synthetic or biologically produced substance which is detectable by humans and intended to appeal to a human aesthetically to create an emotional response, sense of well-being, pleasing perception, physical attraction or arousal. As used herein, the term “pheromone” includes both natural pheromones derived from humans, plants and animals, and synthetic chemical compositions intended to emulate the aesthetic effects of pheromones.

As used herein, the term “activating panel” means a structure which releases a quantity of pheromone by heat, pressure motion or any other means known in the art including non-electronic sensors, electronic sensors, pressure sensors, motion sensors, chemically infused agents activated by heat, motion, skin contact or by any other means known in art

As used herein, the term “pheromone supply reservoir” means a supply container or delivery system which includes a supply of a pheromone for replenishing the pheromone activated area of a garment.

As used herein, the term “tamper resistant opening” refers to a secure opening that, when dosed, is securely dosed and resistant to tampering, such as tearing, but may be easily opened at a point of sale.

As used herein, the term “tamper resistant seam” refers to a secure seam not easily split or unraveled used to secure a pheromone supply reservoir to an article of clothing.

### BACKGROUND

“Pheromone” is a commonly known term used to refer to substances which are secreted or excreted by animals and humans that trigger a social response in members of a target group. Pheromones are chemicals capable of acting outside the body of the secreting individual to impact the behavior of the receiving individual.

Pheromones were first discovered in the late 1960's and identified as strong chemical attractants released by female moths to attract a mate. As more is discovered about these common chemical signals, the definition of pheromone continues to evolve. Scientists now concur that pheromones are not specific to insects or to one species, and that synthetic chemical compounds can have the same effect as naturally occurring pheromones.

The most basic definition for pheromone is any volatile substance that elicits a physiological or endocrine-based change in the receiving organism. Pheromones have been found in organisms ranging from soil nematodes to primates. Ordinarily, pheromones cannot be detected through the normal olfactory system. Instead, pheromones are thought to function through a specialized sensory organ, the vomeronasal organ (VNO).

The VNO is a small, indented patch of membrane that lies just over the Vomer bone in the nose. Receptors in the VNO membrane work in a similar way to regular olfactory receptors, but are highly specific. VNOs in insects, mice, and larger mammals only respond to a few molecules, most of these are specific to the opposite sex of their species. Receptors in the VNO do not send signals into the olfactory bulb of the brain, Instead, receptor cells extend axons into the hypothalamus and amygdala, two “primitive” parts of the brain involved in hormone control and sexual response.

In the 1970's, a Dr. David Berliner discovered a skin extract that, when left open to the air, put his lab workers in uncharacteristically good moods. Following detailed chemical analysis on male and female skin extracts, Dr. Berliner found that two unscented steroid compounds could trigger an electrical response in the human VNO. The two steroid compounds were found to be sex-specific in production as well as detection. Female VNOs were activated only in the presence of the male-specific compound, androstadienone, and male VNO's were similarly specific to the female-specific estratetraenol.

Subsequently, work done by Luis Monti-Bloch at the University of Utah also showed a human behavioral response to these pheromones. Men exposed to less than one millionth of a gram of a synthetic female pheromone became “laid back and relaxed. Their heart and breathing rates slowed, while the capillaries in the skin of their hands dilated, and electrical recordings of the brain found an increase in alpha-wave activity, classic signs of relaxation.

There is no definitive scientific evidence indicating pheromones make a person more or less appealing to the opposite sex. The emotional response that occurs in the presence of human pheromone, then, may simply be the result of associative learning. Although an individual many not consciously smell the natural or synthetic pheromones, the brain associates their presence with pleasurable physical intimacy.

The field of human pheromone research is young and developing, with celebrities such as Paris Hilton endorsing natural and synthetic pheromones and becoming involved in the branding process.

Consumers in the United States and Europe spend over $12 billion dollars annually on perfumes.

It is desirable to accommodate consumers with options for using and pheromones, including clothing choices.

In order for a pheromone to be effective, it must also be reapplied frequently or diffused over time. Chemicals comprising the synthetic or natural pheromone are often heat activated. Perfume manufactures recommend applying pheromone to the warmest parts of the body, which further complicates matters, as the warmest parts of the body of often covered with clothing.

It is desirable to have an apparatus that controls the release of a pheromone over time.

It is further desirable to have an apparatus that maximizes the intensity effect of body heat.

It is further desirable to cross market pheromone and fashion choices in a manner that does not require additional floor or display space and does not make the pheromone vulnerable to theft or tampering.

### SUMMARY OF THE INVENTION

The present invention is a pheromone dispersing clothing apparatus with a body pheromone activating panel adapted to receive pheromone from a pheromone supply reservoir and a receptacle adapted to receive a detachable pheromone supply reservoir that is not accessed until after the garment is purchased.

### DETAILED DESCRIPTION OF INVENTION

For the purpose of promoting an understanding of the present invention, references are made in the text to exemplary embodiments of a fabric pheromone dispersing apparatus, only some of which are described herein. It should be understood that no limitations on the scope of the invention are intended by describing these exemplary embodiments. One of ordinary skill in the art will readily appreciate that alternate but functionally equivalent materials and designs may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

It should be understood that the drawings are not necessarily to scale; instead emphasis has been placed upon illustrating the principles of the invention. In addition, in the embodiments depicted herein, like reference numerals in the various drawings refer to identical or near identical structural elements.

Moreover, the terms “substantially” or “approximately” as used herein may be applied to modify any quantitative representation that could permissibly vary without resulting in a change in the basic function to which it is related.

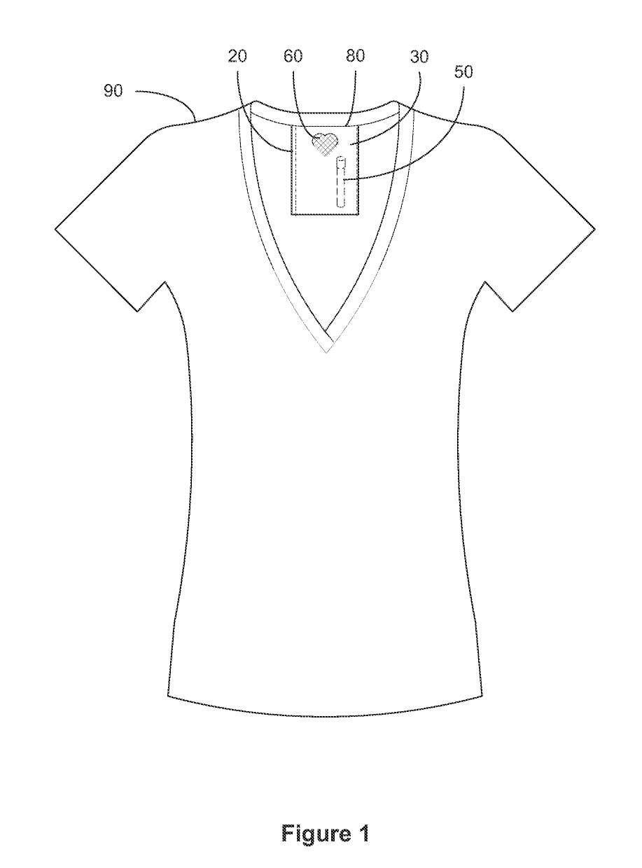

FIG. 1 illustrates an exemplary embodiment of a fabric pheromone dispersing apparatus in use in a garment as the garment is sold at the point-of-purchase, which includes point-of-purchase receptacle 20.

Point-of-purchase receptacle 20 may be a pocket, tether, tube, break-away packaging or any other device in the art which may be used as a point-of-purchase receptacle 20 for a pheromone supply reservoir 50.

In the embodiment shown, point-of-purchase receptacle 20 is a fabric receptacle fixedly attached to the inside back collar of garment 90 by tamper resistant seam 80, which must be cut to access pheromone supply reservoir 50. In the embodiment shown, point-of-purchase. receptacle 20 has outer panel 30 and inner panel 40 (not visible) or any other means known in the art for associating pheromone supply reservoir 50 with a garment at the point-of-purchase or delivery to a consumer. Body pheromone activating panel 60 is located on outer panel 30.

In further exemplary embodiments, point-of-purchase receptacle 20 may contain any number of layers, and layers may be made of different materials.

Body pheromone activating panel 60 is any structure or material to which a substance containing a pheromone can be applied and which is adapted to be positioned in physical contact with the skin of a person wearing garment 90. Heat from the wearer's skin in contact with body pheromone activating panel 60 operates to activate and disperse the applied pheromone in response to body temperature. In the exemplary embodiment shown, body pheromone activating panel 60 is designed and position to be in physical contact with the skin of a person's back or neck. In other embodiments, body pheromone activating panel 60 may be positioned to be in contact with other parts of the body, including pulse points.

In various embodiments, body pheromone activating panel 60 may be of single or composite materials. In still other embodiments, body pheromone activating panel 60 may be of porous or non-porous materials. In various embodiments, body pheromone activating panel 60 may vary in thickness, contours, chemical treatments and shape to control and/or modulate pheromone dispersal.

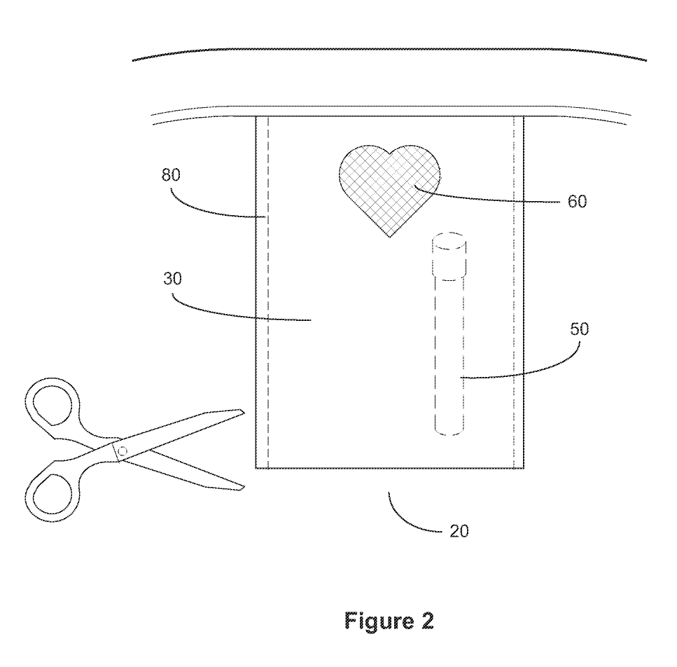

FIG. 2 is an exemplary embodiment of point-of-purchase receptacle 20. In the exemplary embodiment shown, point-of-purchase receptacle 20 is a pouch. Pheromone supply reservoir 50 (e.g., a vial) is located between outer panel 30 and inner panel 40 (not visible). In the embodiment shown, point-of-purchase receptacle 20 includes tamper resistant seam 80 and pheromone supply reservoir 50. Tamper resistant seam 80 attaches point-of-purchase receptacle 20 to a shirt or other garment (not shown). In the embodiment shown, point-of-purchase receptacle 20 may be cut after purchase to remove pheromone supply reservoir 50.

In further exemplary embodiments, point-of-purchase receptacle 20 may be adapted to hold more or fewer pheromone supply reservoirs 50, and pheromone supply reservoir 50 and body pheromone activating panel 60 may be adapted to hold different pheromone formulations and pheromones in different physical states, such as gels, powders, bars, creams, crystals, and other absorbent or soluble materials.

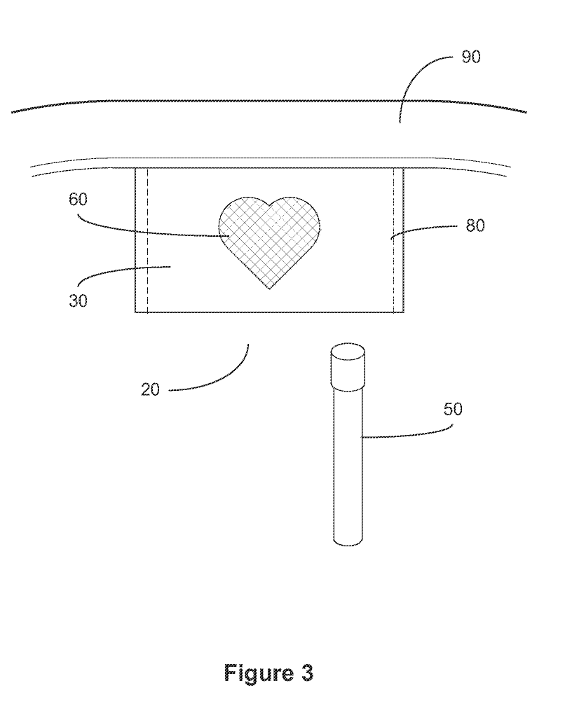

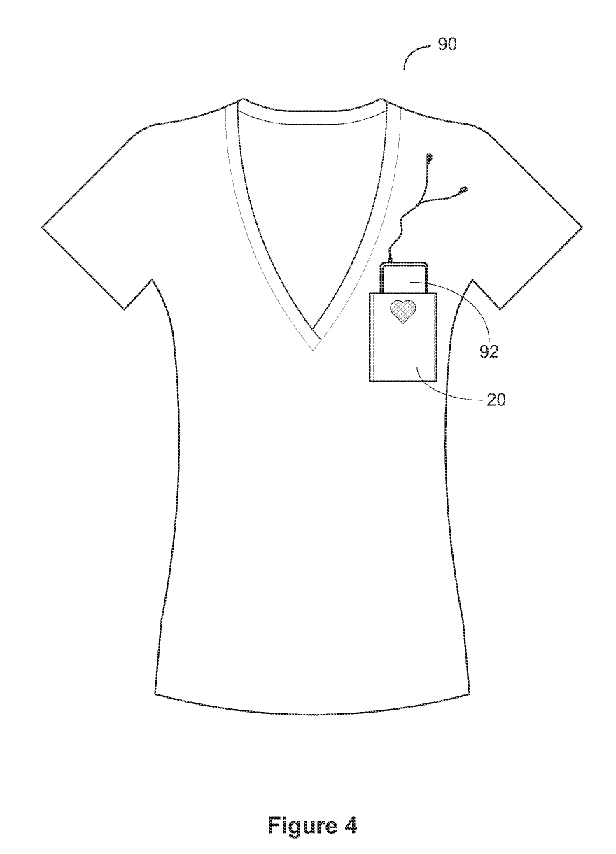

FIG. 3 illustrates an exemplary embodiment of a pheromone dispersing clothing apparatus ready for use. Body pheromone activating panel 60, still attached to garment 90, is exposed, and positioned and designed to be in physical contact with a person's skin when garment 90 is worn. The remaining portions of point-of-purchase receptacle 20 have been removed to release pheromone supply reservoir 50. Pheromone from pheromone supply reservoir 50 is dabbed on body pheromone activating panel 60. Body pheromone activating panel 60 disperses pheromone when activated by body heat. In the embodiment shown, body pheromone activating panel 60 is designed and positioned to be in physical contact with the skin of a person's neck.

In further exemplary embodiments, point-of-purchase receptacle 20 may be partially removed so that the remaining body pheromone activating panel 60 is unobtrusive. In yet further embodiments, body pheromone activating panel 60 may also be in a different position on outer panel 30, so that more point-of-purchase receptacle 20 may be removed. In still further embodiments, body pheromone activating panel 60 may be designed and positioned to be in physical contact with a person's pulse point or other area of skin. In still other embodiments, body pheromone activating panel 60 may protrude from garment 90 or be otherwise visible on the exterior of garment 90 as an aesthetic component of garment 90.

In the exemplary embodiments described in FIGS. 1-3, pheromone supply reservoir 50 is a breakable structure which is removable from garment 90 and replaceable. In further exemplary embodiments, pheromone supply reservoir 50 may be reusable.

FIG. 4 illustrates an exemplary embodiment of a pheromone dispersing apparatus activated by heat generated from a personal electronic device. As illustrated in FIG. 4, point-of-purchase receptacle 20 is illustrated as a pocket or pouch on garment 90, approximately the size of an mp3 player, cell phone or other personal electronic device. When a pheromone is applied to body pheromone activating panel 60, heat generated from personal electronic device 92 activates the pheromone and causes it to be dispersed.

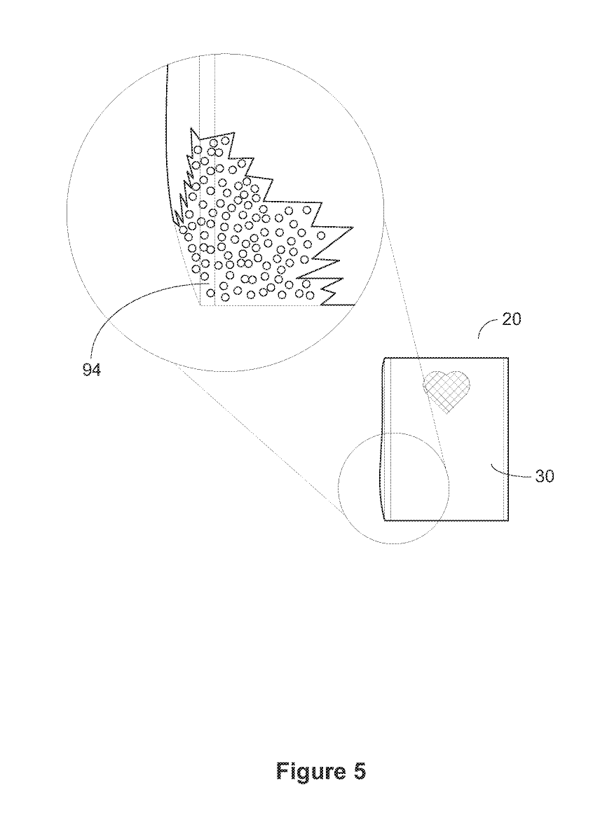

FIG. 5 illustrates an exemplary embodiment of a pheromone dispersing apparatus activated by iron oxide. As illustrated in FIG. 5, point-of-purchase receptacle 20 contains a quantity of iron powder mixture 94 located between outer panel 30 and inner panel 40 (not shown). Upon exposure to oxygen, iron powder mixture 94 reacts with oxygen to release heat.

In the exemplary embodiment shown, iron powder mixture 94 is a mixture of iron powder, salt, water, an absorbent material and activated carbon, similar to the mixture found in standard hand warmers. When the iron powder is in contact with oxygen in the presence of salt and water, the oxygen reacts with the iron powder to form iron oxide and release heat. In further exemplary embodiments, iron powder mixture 94 may be any mixture known in the art to react with oxygen, or other elements in air, in an exothermic reaction. In still further exemplary embodiments, iron powder mixture 94 may be any mixture or combination of mixtures, separately or jointly packaged, which react in an exothermic reaction. The heat generated by any exothermic reaction, however, should not be great enough to burn a wearer of the garment or the garment itself.

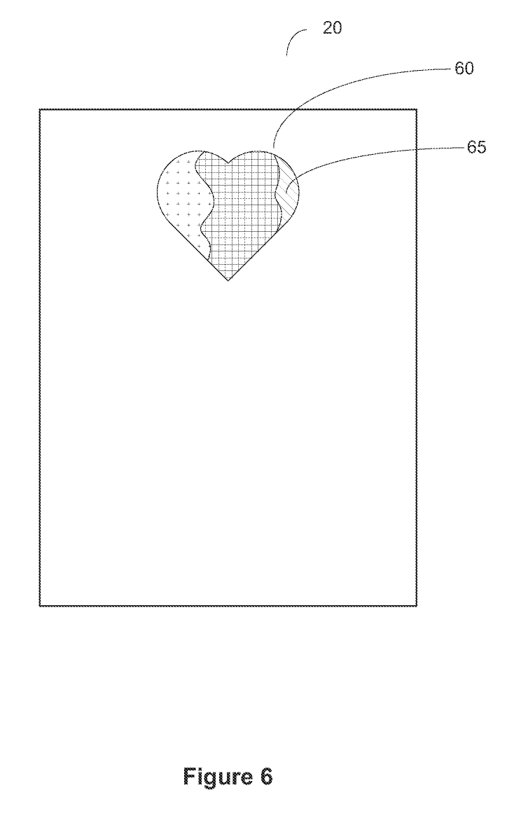

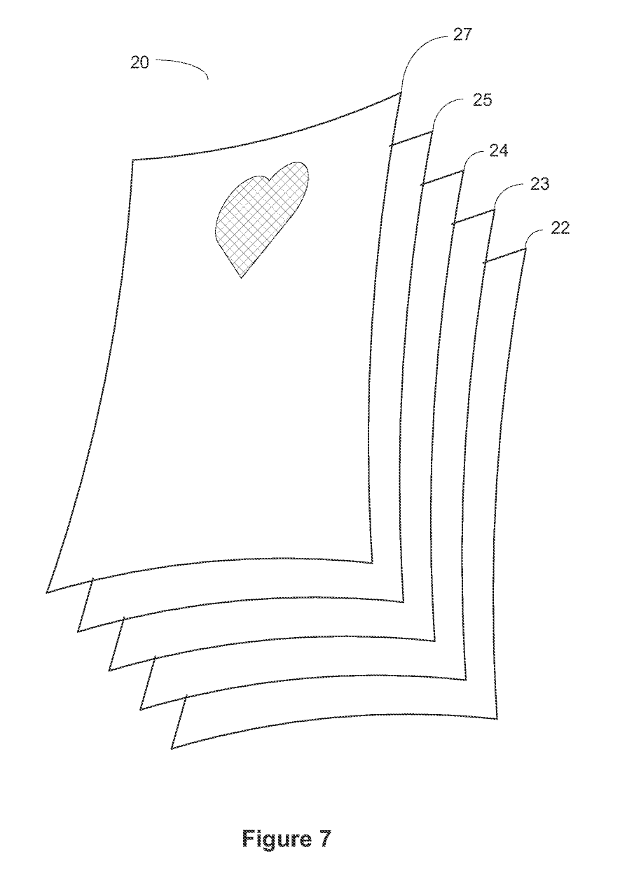

FIG. 6 illustrates an exemplary embodiment of a pheromone dispersing apparatus which indicates the presence of pheromones with a thermochromatic textile area. In the exemplary embodiment shown, body pheromone activating panel 60 contains thermochromatic textile area 65, which changes color when activated by heat. In further exemplary embodiments, thermochromatic textile area 65 may be located anywhere on point-of-purchase receptacle.

As illustrated in FIG. 6, thermochromatic textile area 65 is partially activated, or changed. In further exemplary embodiments, thermochromatic textile area 65 may be any material known in the art to react to changes in temperature, such as heat-sensitive materials. For example, thermochromatic textile area 65 may react to the application of a liquid or gel pheromone which causes a temperature change.

In still further exemplary embodiments, thermochromatic textile area 65 may also react to the application of moisture.

In further exemplary embodiments, body pheromone activating panel 60 may contain any stimuli sensitive material or materials, such as polymers or compounds which change size, shape or other physical property based on temperature, humidity, UV light, light, and other outside stimuli.

FIG. 7 illustrates the Multiple layers of an exemplary pheromone dispersing apparatus. In the exemplary embodiment shown, point-of-purchase receptacle contains multiple layers, including satin layer 22, non-absorbent layer 23, absorbent layer 24, duck cloth layer 25 and thermochromatic material area 27. As illustrated in FIG. 7, non-absorbent layer 23 is made of webbed pvc and provides a structural barrier to prevent moisture from entering satin layer 22. In further exemplary embodiments, non-absorbent layer 23 may be any non-absorbent material known in the art, such as netted fibers.

In the exemplary embodiment shown, absorbent layer 24 is cotton. In further exemplary embodiments, absorbent layer 24 may be any absorbent material known in the art, including wax-based solids. For example, absorbent layer 24 may be a dispersible heat-sensitive, oil-based solid infused with a pheromone. In still further exemplary embodiments, absorbent layer 24 may contain portions of different absorbent materials having different absorptive properties.

As illustrated in FIG. 7, layers 22, 23, 24, 25 and 27 are the same size as point-of-purchase receptacle 20 with satin layer 22 forming outer panel 30 and thermochromatic material area 27 forming inner panel 40. In further exemplary embodiments, layers 22, 23, 24, 25 and 27 may be of different sizes and may correspond only to body pheromone activating panel 60. In still further exemplary embodiments, outer panel 30 and inner panel 40 may be omitted, or a layer or layers from layers 22, 23, 24, 25 and 27 may be omitted.

In further exemplary embodiments, any of layers 22, 23, 24, 25 and 27 may be selectively attachable, and, in some exemplary embodiments, point-of-purchase receptacle 20 may be selectively attachable.

What is claimed is:

1. A pheromone dispersing apparatus comprised of:
at least one garment which includes an activating panel adapted to receive a quantity of pheromone from a pheromone supply reservoir; and
at least one detachable pheromone supply reservoir.

2. The apparatus of claim 1 wherein said pheromone supply reservoir is a spray vial.

3. The apparatus of claim 1 wherein said pheromone supply reservoir is a breakable glass vial.

4. The apparatus of claim 1 wherein said activating panel further includes a heat sensitive patch.

5. The apparatus of claim 1 wherein said activating panel includes a thermochromatic textile which changes color to indicate the application of said quantity of pheromone.

6. The apparatus of claim 1 wherein said activating panel includes a thermochromatic textile which changes color to indicate the presence of heat.

7. The apparatus of claim 1 wherein said activating panel is comprised of at least of absorbent material and contains at least one area of non-absorbent material.

8. The apparatus of claim 7 wherein said absorbent material is cotton.

9. The apparatus of claim 7 wherein said non-absorbent material is netted fiber.

10. The apparatus of claim 1 wherein said activating panel is compromised of a satin layer, a cotton layer, a webbed pvc layer, and a thermochromatic textile area, wherein said webbed pvc layer provides a structural barrier to prevent moisture from entering said satin layer.

11. The apparatus of claim 1 wherein said activating panel is activated by heat from a personal electronic device.

12. The apparatus of claim 1 wherein said activating panel is comprised of a satin layer and a pvc fabric layer, wherein said satin layer forms a pouch.

13. The apparatus of claim 1 wherein said activating panel is comprised of two satin layers having dimensions of between three and five inches and joined by a seam, a pvc moisture barrier, and a material layer containing a thermochromatic textile fabric area.

14. A pheromone dispersing apparatus comprised of:
a pheromone activating pouch adapted to receive a quantity of pheromone from
a pheromone supply reservoir comprised of
a satin layer,
a pvc backing,
an absorbent layer,
a duck cloth layer, and
a thermochromatic material area; and
at least one detachable pheromone supply reservoir.

15. The apparatus of claim 14 wherein said pouch contains a quantity of iron powder mixture.

16. The apparatus of claim 14 wherein said pouch contains a reservoir of stimuli sensitive materials, wherein said stimuli sensitive materials are polymer materials that are able to change size, shape or other physical characteristic based on outside stimuli.

17. The apparatus of claim 14 wherein said pouch contains multiple patches of different absorbent layers.

18. The apparatus of claim 14 wherein at least one of said satin layer, pc backing, absorbent layer, duck cloth layer and thermochromatic material area is selectively attachable.

19. The apparatus of claim 14 wherein said absorbent layer is a dispersible heat-sensitive, oil-based solid infused with a pheromone.

20. The apparatus of claim 14 wherein said absorbent layer is wax-based.
